(19) United States
(12) Patent Application Publication
(10) Pub. No.: US 2009/0119801 A1
(43) Pub. Date: May 7, 2009

(54) Method for preserving cut flowers, cut flower preservation kit, method for manufacturing processed cut flowers, and processed cut flowers

(75) Inventors: Masaki Hashimoto, Osaka (JP); Takahiro Hayashi, Uji-shi (JP)

Correspondence Address: DRINKER BIDDLE & REATH (DC), 1500 K STREET, N.W., SUITE 1100, WASHINGTON, DC, 20005-1209, US

(73) Assignee: SUNTORY FLOWERS LIMITED

(21) Appl. No.: 11/902,360

(22) Filed: Sep. 20, 2007

(51) Int. Cl. A01N 3/02; A01H 5/02

(52) U.S. Cl. 800/298; 504/114

(57) ABSTRACT

A method for preserving a cut flower involving only an easy and simple procedure, whereby the cut flower can maintain qualities similar to those of the natural state for a long period of time, and a method for manufacturing a processed cut flower that can maintain qualities similar to those of the natural state for a long period of time. The method for preserving a cut flower by replacing the tissue fluid of the cut flower by a preservative solution includes the steps of: (1) a first replacement step in which the tissue fluid of the cut flower is replaced by a hydrophilic organic solvent; and (2) a second replacement step in which the hydrophilic organic solvent having replaced the tissue fluid in the cut flower is replaced by the preservative solution of an organic solvent that has an affinity for the hydrophilic organic solvent and is nonvolatile or hardly-volatile.

### BACKGROUND OF THE INVENTION

1. Field of the Invention

The present invention relates to a method for preserving a cut flower by replacing the tissue water of the cut flower by a preservative solution so that the cut flower can be preserved in a fresh state for an extended period of time, and a method for manufacturing a processed cut flower that can be preserved in a fresh state for an extended period of time.

2. Description of Related Art

In recent years, processed flowers called preserved flowers are attracting a lot of attention. The preserved flowers are flowers obtained by replacing the water contained in the tissues (hereinafter referred to as “tissue fluid”) of a cut flower such as a rose or carnation by a preservative solution. Because preserved flowers have qualities (shape, texture, color, etc.) very similar to those of the natural state, as compared to conventional dried flowers, preserved flowers are often used, as a unique flower material, for wedding bouquets and for flower arrangements for decorating the entrances and living rooms of ordinary households.

As a method for producing such preserved flowers, there is a method in which polyethylene glycol or the like is used as a preservative solution (see Japanese Patent No. 3548744).

However, the preserved flowers produced by the method described in the above patent document deteriorate rapidly, particularly under conditions of high temperatures and high humidity, and the petals turn white after several days, some of which may become transparent. In addition, the preserved flowers are required to be recolored by adding an artificial coloring agent to the preservative solution, or the like. Accordingly, the method does not always provide excellent reproducibility and stability.

The present invention has been accomplished under the circumstances described above, and the present invention provides a method for preserving a cut flower involving only an easy and simple procedure, whereby the cut flower can maintain qualities similar to those of the natural state for a long period of time, and a method for manufacturing a processed cut flower that can maintain qualities similar to those of the natural state for a long period of time.

### SUMMARY OF THE INVENTION

A first characteristic means of a method for preserving a cut flower according to the present invention lies in a method for preserving a cut flower by replacing tissue fluid of the cut flower by a preservative solution, the method including: (1) a first replacement step in which the tissue fluid of the cut flower is replaced by a hydrophilic organic solvent; and (2) a second replacement step in which the hydrophilic organic solvent that has replaced the tissue fluid in the cut flower is replaced by a preservative solution comprising an organic solvent that has an affinity for the hydrophilic organic solvent and is nonvolatile or hardly-volatile.

### [Action and Effect]

In the present invention, because the hydrophilic organic solvent is used, the tissue fluid of the cut flower can be rapidly replaced by the hydrophilic organic solvent, and the cut flower can be dehydrated.

Further, the organic solvent used as the preservative solution in the present invention has an affinity for the hydrophilic organic solvent, and thus the hydrophilic organic solvent contained in the cut flower can be rapidly replaced by the organic solvent. In addition thereto, because the organic solvent is nonvolatile or hardly-volatile, contraction and deformation of the petals hardly occur, and therefore the shape of the cut flower can be maintained for a long period of time.

Furthermore, by replacing the tissue fluid of the cut flower by the preservative solution (organic solvent), the tissues of the cut flower will be fixed or the like, making it unlikely to develop decay or the like. As a result, the texture and color of the cut flower can be maintained for a long period of time.

Therefore, the cut flower processed according to the present invention can maintain qualities (shape, texture, color, etc.) similar to those of the original natural state.

The finding that, by subjecting a cut flower to the easy and simple process including a first replacement step using a hydrophilic solvent and a second replacement step using an organic solvent that has an affinity for the hydrophilic organic solvent and is nonvolatile or hardly-volatile, the cut flower can maintain qualities (shape, texture, color, etc.) similar to those of the original natural state for a long period of time is noteworthy in that it is found for the first time by the present inventors through their extensive studies.

A second characteristic means of the method for preserving a cut flower according to the present invention lies in that the preservative solution is at least one selected from the group consisting of 2-methyl-2,4-pentanediol, 2-ethyl-1,3-hexanediol, 2-[2-(2-ethylhexyloxy)ethoxy]ethanol, 3-methyl-1,5-pentanediol, 2-(2-ethylhexyloxy)ethanol and polypropylene glycol.

### [Action and Effect]

As shown in Example 2, which will be described later, with the use of the preservative solution such as 2-methyl-2,4-pentanediol, 2-ethyl-1,3-hexanediol, 2-[2-(2-ethylhexyloxy)ethoxy]ethanol, 3-methyl-1,5-pentanediol, 2-(2-ethylhexyloxy)ethanol or polypropylene glycol, the cut flower can maintain qualities (shape, texture, color, etc.) similar to those of the original natural state for a long period of time. Particularly, these preservative solutions hardly allow the natural pigment (anthocyanin, etc.) contained in the cut flower to leach out during the second replacement step, and thus the natural pigment is likely to be kept in the cut flower. For this reason, the cut flower can maintain a color similar to or substantially identical to that of the original natural state.

The preservative solution usable in the present invention may be one selected from 2-methyl-2,4-pentanediol, 2-ethyl-1,3-hexanediol, 2-[2-(2-ethylhexyloxy)ethoxy]ethanol, 3-methyl-1,5-pentanediol, 2-(2-ethylhexyloxy)ethanol and polypropylene glycol, or may be a mixture prepared by mixing a plurality of solvents, which are selected as appropriate from the above solvents.

A third characteristic means of the method for preserving a cut flower according to the present invention lies in that the hydrophilic organic solvent includes at least one liquid selected from the group consisting of ethanol, 1,4-dioxane, n-butyric acid, acetone and 1-propanol.

### [Action and Effect]

Because the hydrophilic organic solvents such as ethanol, 1,4-dioxane, n-butyric acid, acetone and 1-propanol have an affinity for water, the replacement (dehydration) occurs easily.

Further, as shown in Example 1, which will be described later, when a cut flower is immersed in the hydrophilic organic solvent, the hydrophilic organic solvent hardly allows the natural pigment contained in the cut flower to leach out, and makes it difficult for the cut flower to undergo contraction.

These properties that the hydrophilic organic solvents possess are found for the first time by the present inventors through their extensive studies.

The hydrophilic organic solvent usable in the present invention may be one selected from ethanol, 1,4-dioxane, n-butyric acid, acetone and 1-propanol, or may be a mixture prepared by mixing a plurality of solvents, which are selected as appropriate from the above solvents.

A fourth characteristic means of the method for preserving a cut flower according to the present invention lies in that a washing step is further included in which the surface of the cut flower having been subjected to the second replacement step is rinsed with a further hydrophilic organic solvent than said hydrophilic organic solvent.

### [Action and Effect]

The cut flower after having been subjected to the second replacement step may exhibit unnatural color and texture due to the preservative solution adhering to the surface of the cut flower.

For this reason, according to the present invention, the preservative solution adhering to the surface of the cut flower is washed off by rinsing with a hydrophilic organic solvent, whereby the color and texture of the cut flower can be made similar to those of the original natural state.

A characteristic configuration of a cut flower preservation kit according to the present invention lies in that the kit is usable to perform the method for preserving a cut flower that includes the above-described characteristic means, and the kit includes at least the hydrophilic organic solvent and the preservative solution.

### [Action and Effect]

With the use of the cut flower preservation kit of the present invention, the method for preserving a cut flower that includes the above-described characteristic means can be performed in an easy and simple manner.

A first characteristic means of a method for manufacturing a processed cut flower according to the present invention lies in a method for manufacturing a processed cut flower by replacing tissue fluid of a cut flower by a preservative solution, the method including: (1) a first replacement step in which the tissue fluid of the cut flower is replaced by a hydrophilic organic solvent; and (2) a second replacement step in which the hydrophilic organic solvent having replaced the tissue fluid in the cut flower is replaced by a preservative solution comprising an organic solvent that has an affinity for the hydrophilic organic solvent and is nonvolatile or hardly-volatile.

### [Action and Effect]

In the present invention, because the hydrophilic organic solvent is used, the tissue fluid of the cut flower can be rapidly replaced by the hydrophilic organic solvent, and the cut flower can be dehydrated.

Further, the organic solvent used as the preservative solution in the present invention has an affinity for the hydrophilic organic solvent, and thus the hydrophilic organic solvent contained in the cut flower can be rapidly replaced by the organic solvent. In addition thereto, because the organic solvent is nonvolatile or hardly-volatile, contraction and deformation of the petals hardly occur, and therefore the shape of the cut flower can be maintained for a long period of time.

Furthermore, by replacing the tissue fluid of the cut flower by the preservative solution (organic solvent), the tissues of the cut flower will be fixed or the like, making it unlikely to develop decay or the like. As a result, the texture and color of the cut flower can be maintained for a long period of time.

Therefore, the processed cut flower manufactured by the present invention can maintain qualities (shape, texture, color, etc.) similar to those of the original natural state.

The finding that, with the easy and simple process including a first replacement step using a hydrophilic solvent and a second replacement step using an organic solvent that has an affinity for the hydrophilic organic solvent and is nonvolatile or hardly-volatile, a processed cut flower can be manufactured that can maintain qualities (shape, texture, color, etc.) similar to those of the original natural state for a long period of time is noteworthy in that it is found for the first time by the present inventors through their extensive studies.

A second characteristic means of the method for manufacturing a processed cut flower according to the present invention lies in that the preservative solution is at least one selected from the group consisting of 2-methyl-2,4-pentanediol, 2-ethyl-1,3-hexanediol, 2-[2-(2-ethylhexyloxy)ethoxy]ethanol, 3-methyl-1,5-pentanediol, 2-(2-ethylhexyloxy)ethanol and polypropylene glycol.

### [Action and Effect]

As shown in Example 2, which will be described later, with the use of the preservative solution such as 2-methyl-2,4-pentanediol, 2-ethyl-1,3-hexanediol, 2-[2-(2-ethylhexyloxy)ethoxy]ethanol, 3-methyl-1,5-pentanediol, 2-(2-ethylhexyloxy)ethanol or polypropylene glycol, the cut flower can maintain qualities (shape, texture, color, etc.) similar to those of the original natural state for a long period of time. Particularly, these preservative solutions hardly allow the natural pigment (anthocyanin, etc.) contained in the cut flower to leach out during the second replacement step, and thus the natural pigment is likely to be kept in the cut flower. For this reason, the cut flower can maintain a color similar to or substantially identical to that of the original natural state.

The preservative solution usable in the present invention may be one selected from the group consisting of 2-methyl-2,4-pentanediol, 2-ethyl-1,3-hexanediol, 2-[2-(2-ethylhexyloxy)ethoxy]ethanol, 3-methyl-1,5-pentanediol, 2-(2-ethylhexyloxy)ethanol and polypropylene glycol, or may be a mixture prepared by mixing a plurality of solvents, which are selected as appropriate from the above solvents.

A third characteristic means of the method for manufacturing a processed cut flower according to the present invention lies in that the hydrophilic organic solvent includes at least one liquid selected from ethanol, 1,4-dioxane, n-butyric acid, acetone and 1-propanol.

### [Action and Effect]

Because the hydrophilic organic solvents such as ethanol, 1,4-dioxane, n-butyric acid, acetone and 1-propanol have an affinity for water, the replacement (dehydration) occurs easily.

Further, as shown in Example 1, which will be described later, when a cut flower is immersed in the hydrophilic organic solvent, the hydrophilic organic solvent hardly allows the natural pigment contained in the cut flower to leach out, and makes it difficult for the cut flower to undergo contraction.

These properties that the hydrophilic organic solvents possess are found for the first time by the present inventors through their extensive studies.

The hydrophilic organic solvent used in the present invention may be one selected from ethanol, 1,4-dioxane, n-butyric acid, acetone and 1-propanol, or may be a mixture prepared by mixing a plurality of solvents, which are selected as appropriate from the above solvents.

A fourth characteristic means of the method for manufacturing a processed cut flower according to the present invention lies in that a washing step is further included in which the surface of the cut flower having been subjected to the second replacement step is rinsed with a further hydrophilic organic solvent than said hydrophilic organic solvent.

### [Action and Effect]

The cut flower after having been subjected to the second replacement step may exhibit unnatural color and texture due to the preservative solution adhering to the surface of the cut flower.

For this reason, according to the present invention, the preservative solution adhering to the surface of the cut flower is washed off by rinsing with a hydrophilic organic solvent, whereby the color and texture of the cut flower can be made similar to those of the original natural state.

A characteristic configuration of a processed cut flower according to the present invention lies in that the processed cut flower is manufactured by the method for manufacturing a processed cut flower that includes the above-described characteristic means.

### [Action and Effect]

The processed cut flower of the present invention can maintain qualities (shape, texture, color, etc.) similar to those of the original natural state for a long period of time.

### DETAILED DESCRIPTION OF THE INVENTION

Hereinafter, embodiments of the present invention will be described.

### Embodiment

The present invention includes (1) a first replacement step and (2) a second replacement step, and may further include (3) a washing step and (4) a drying step as appropriate, which will be described below.

### (1) First Replacement Step

First, the tissue fluid contained in the tissues of a cut flower is replaced by a hydrophilic organic solvent, which will be described below. For example, a prepared cut flower is immersed in a container containing a hydrophilic organic solvent for a predetermined period of time. It should be noted that the replacement method is not limited to the immersion.

### (Hydrophilic Organic Solvent)

“Hydrophilic organic solvent” as used in the present invention means an organic solvent that has at least an affinity for water and a property that it can replace the tissue fluid of a cut flower. It is preferable that such a hydrophilic organic solvent further has properties that it hardly allows the natural pigment contained in the cut flower to leach out and hardly cause contraction of the cut flower during the first replacement step in which the cut flower is immersed in the hydrophilic organic solvent. More specifically, it is preferable to use a hydrophilic organic solvent that has a partition coefficient logP (a parameter that indicates whether the molecule is hydrophilic or hydrophobic) of around −0.271 to 0.344, and a low viscosity.

Typical examples of the hydrophilic organic solvent applicable in the present invention include ethanol, 1,4-dioxane, n-butyric acid, isobutyric acid, acetone, 1-propanol, 2-propanol, methanol, 1-butanol and 2-butanol. They can be selected as appropriate according to the type of cut flower or the like, but it is more preferable to use ethanol, 1,4-dioxane, n-butyric acid, acetone or 1-propanol, and it is most preferable to use ethanol.

### (2) Second Replacement Step

Subsequently, the hydrophilic organic solvent contained in the tissues of the cut flower is replaced by a preservative solution, which will be described below. For example, the cut flower dehydrated in the first replacement step is immersed in a container containing a preservative solution. It should be noted that the replacement method is not limited to the immersion.

### (Preservative Solution)

“Preservative solution” as used in the present invention means an organic solvent that has at least an affinity for the above hydrophilic organic solvent and is nonvolatile or hardly-volatile. It is preferable that such a preservative solution further has a property that it hardly allows the natural pigment contained in the cut flower to leach out. More specifically, it is preferable to use a preservative solution that has a partition coefficient logP (a parameter that indicates whether the molecule is hydrophilic or hydrophobic) of around −0.236 to 2.8266, and is in a liquid state at room temperature, because it provides excellent preservability of the shape of the petals.

Typical examples of the preservative solution applicable in the present invention include 2-methyl-2,4-pentanediol, 2-ethyl-1,3-hexanediol, 2-[2-(2-ethylhexyloxy)ethoxy]ethanol, 3-methyl-1,5-pentanediol, 2-(2-ethylhexyloxy)ethanol and polypropylene glycol. They can be selected as appropriate according to the type of cut flower or the like, but it is preferable to use polypropylene glycol or 2-methyl-2,4-pentanediol.

### (3) Washing Step

After the second replacement step, the surface of the cut flower is lightly rinsed with a cleaning solution, which will be described below, so as to wash off the preservative solution adhering to the surface. This washing step is effective, particularly when a nonvolatile organic solvent is used as the preservative solution in the second replacement step, for removing the nonvolatile organic solvent adhering to the surface of the cut flower.

### (Cleaning Solution)

As the cleaning solution usable in the present invention, any solvent except water can be used as long as the preservative solution adhering to the surface of the cut flower can be washed off.

As the cleaning solution, for example, a hydrophilic organic solvent such as ethanol, 1,4-dioxane, n-butyric acid, isobutyric acid, acetone, 1-propanol, 2-propanol, methanol, 1-butanol or 2-butanol is preferably used, and it is particularly preferable to use a volatile hydrophilic organic solvent.

### (4) Drying Step

Finally, the cut flower after having been rinsed in the washing step is dried to remove the cleaning solution remaining on the surface of the cut flower. As the drying method, drying under mild temperature conditions that does not affect the qualities of the cut flower (processed cut flower) after having been subjected to the above-described processes is preferable such as leaving it at room temperature, air-drying and drying by applying worm air. The drying time can be set as appropriate within a range from about several minutes to several weeks.

### (Cut Flowers)

The present invention is applicable to various flowers including, but not limited to, carnation, rose, chrysanthemum, lily, orchid, gerbera, sunflower, delphinium, gentian, commelinaceae, morning glory, dahlia, amaryllis, oxypetalum, oncidium, alstromeria, eustoma russellianum, dendrobium phalaenopsis, petunia, globe amaranth and sweet pea.

### (Processed Cut Flowers)

“Processed cut flower” as used in the present invention refers to a processed product of a cut flower obtained by subjecting the cut flower to the above-described first replacement step and second replacement step, and optionally the above-described washing step and drying step. In the processed cut flower of the present invention, the water that the original flower contained is replaced by the preservative solution, and thus the processed cut flower of the present invention has a feature of an improved preservability as compared to natural flowers.

### (Tissue Fluid)

“Tissue fluid” as used in the present invention means a liquid (mainly water) that is present in the tissues of a cut flower.

### Other Embodiments

1. The embodiments of the present invention are not limited to those described above, and it is also possible to add a step of recoloring the cut flower by adding an artificial pigment to the preservative solution, where appropriate.

2. The present invention may be carried out by using a cut flower preservation kit including at least the hydrophilic organic solvent and the preservative solution, which are contained in appropriate containers.

### EXAMPLE

Hereinafter, the present invention will be described by way of examples, but it should be understood that the present invention is not limited thereto.

### Example 1

### Investigation of Hydrophilic Organic Solvent Applicable in the Present Invention

As a cut flower, a carnation named “Moondust Velvet Blue” (available from Suntory Flowers Ltd.) was used. The petals of the carnation were immersed in various organic solvents listed in Table 1 for not less than one day, after which a determination was made as to whether the water contained in the tissues of the petals (hereinafter referred to as “tissue fluid”) was replaced by the organic solvent, and whether the petals were contracted. The leach-out of the pigment was evaluated by measuring the amount of the pigment remaining in the petals according to the following measurement method. The results are shown in Table 1. The test confirmed that all the organic solvents used had replaced the tissue fluid. Accordingly, it was concluded that these organic solvents were possibly usable as the hydrophilic organic solvent in the first replacement step.

The hydrophilic organic solvent used in the present invention can be selected as appropriate according to the type and color of a raw cut flower, taking into consideration the degree of the leach-out of the pigment and the degree of contraction of the shape of the petals, but in this example in which the carnation “Moondust Velvet Blue” was used as a cut flower, when the leach-out of the pigment and the occurrence of the contraction were considered, it was found that ethanol, 1,4-dioxane, n-butyric acid, acetone and 1-propanol were suitable as the hydrophilic organic solvent.

### <Method for Measuring Amount of Remaining Pigment>

1. The purple portions of the petals in an amount of 0.15 g were immersed in the organic solvent for about one day.

2. The petals after the above process 1 were immersed in another test tube containing 10 mL of methanol-hydrochloric acid (with a hydrochloric acid volume ratio of 1/35, that is, hydrochloric acid:methanol=1:34) for about one day.

3. Using an absorptiometer (U-2000A, a double beam spectrophotometer available from Hitachi Ltd.), the peak value at around 540 nm was measured, which was denoted as the amount of remaining pigment.

TABLE 1

| organic | partition | boiling point | replace- | leach-out of | contrac- |
|---|---|---|---|---|---|
| solvent | coefficient | (° C.) | ment | pigment | tion |
| ethanol | −0.187 | 78 | yes | no | no |
| 1-propanol | 0.344 | 97 | yes | no | no |
| n-butyric | 0.777 | 162 | yes | no | no |
| acid | | | | | |
| 1,4-dioxane | −0.271 | 100 to 102 | yes | no | no |
| acetone | −0.157 | 56 | yes | no | no |
| 2-propanol | | 82 | yes | variation | no |
| | | | | occurred | |
| Methanol | −0.719 | 64.7 | yes | yes | yes |
| 1-butanol | 0.875 | 116 to 118 | yes | no | yes |
| 2-butanol | 0.691 | 98 | yes | variation | no |
| | | | | occurred | |

### Example 2

### Investigation of Preservative Agent Applicable in the Present Invention

As a cut flower, a carnation named “Moondust Velvet Blue” (available from Suntory Flowers Ltd.) was used. The petals of the carnation were immersed in various organic solvents listed in Table 2, and a determination was made as to whether a contraction (hereinafter referred to as “contraction A”) occurred. After the petals were immersed in each organic solvent for not less than one day, a determination was made as to whether a variation occurred in the color tone of the petals, the color of the petals, the shine of the petals, and whether a contraction (hereinafter referred to as “contraction B”) occurred in the petals during the process in which the organic solvent was volatilized from the petals. The results are shown in Table 2. As a result of the test, with any of the organic solvents, no contraction occurred in the petals. Accordingly, it was concluded that those organic solvents were possibly usable as the preservative solution in the present invention.

The preservative solution used in the present invention can be selected as appropriate according to the type and color of a raw cut flower, taking into consideration the degree of the leach-out of the pigment and the degree of contraction of the shape of the petals, but in this example in which the carnation “Moondust Velvet Blue” was used as a cut flower, when the color of the petals and the like were considered, it was found that 2-methyl-2,4-pentanediol, 2-ethyl-1,3-hexanediol, 2-[2-(2-ethylhexyloxy)ethoxy]ethanol, 3-methyl-1,5-pentanediol, 2-(2-ethylhexyloxy)ethanol, diol type polypropylene glycol 400 (available from Wako Pure Chemical Industries, Ltd.), diol type polypropylene glycol 700 (available from Wako Pure Chemical Industries, Ltd.) were suitable as the preservative solution.

TABLE 2

| | boiling | volatilization | contraction | contraction | | color of | shine |
|---|---|---|---|---|---|---|---|
| organic solvent | point (° C.) | rate (%) | A | B | variation in color tone | petals | of petal |
| 2-methyl-2,4-pentanediol | 197 | 3 | no | no | uniform color tone | dark purple | no, |
| | | | | | | | wetted |
| 2-ethyl-1,3-hexanediol | 241 to 249 | 0 | no | no | uniform color tone | dark purple | yes |
| 2-[2-(2-ethylhexyloxy)ethoxy]ethanol | 229 | 0 | no | no | not greater than 20% | light purple | yes |
| | | | | | of non-uniform area | | |
| 3-methyl-1,5-pentanediol | 216 | 0 | no | no | not greater than 20% | purple | yes |
| | | | | | of non-uniform area | | |
| 2-(2-ethylhexyloxy)ethanol | 229 | 1 | no | no | not greater than 20% | purple | no |
| | | | | | of non-uniform area | | |
| diol type polypropylene glycol 400 | | 0 | no | no | uniform color tone | dark purple | yes |
| diol type polypropylene glycol 700 | | 0 | no | no | uniform color tone | dark purple | yes |
| glycerin | 182 | 0 | no | no | not greater than 20% | colorless, | yes |
| | | | | | of non-uniform area | transparent | |
| ethylene glycol | 196 to 198 | 0 | no | no | not greater than 20% | colorless, | yes |
| | | | | | of non-uniform area | transparent | |
| | | | | | | (very light | |
| | | | | | | purple) | |

### Example 3

### Affinity Between Hydrophilic Organic Solvent and Preservative Solution

Evaluation was made for the affinity between the hydrophilic organic solvents (ethanol, 1,4-dioxane, n-butyric acid, acetone and 1-propanol) and the preservative solutions (2-methyl-2,4-pentanediol, 2-ethyl-1,3-hexanediol, 2-[2-(2-ethylhexyloxy)ethoxy]ethanol, 3-methyl-1,5-pentanediol, 2-(2-ethylhexyloxy)ethanol, diol type polypropylene glycol 400 (available from Wako Pure Chemical Industries, Ltd.) and diol type polypropylene glycol 700 (available from Wako Pure Chemical Industries, Ltd.)). Each of the above hydrophilic organic solvents and each of the above preservative solutions were mixed, and the state of the mixture was observed. As a result, all the mixtures of the above combinations were uniform and no separation was observed, and therefore it was confirmed that there was an affinity between the hydrophilic organic solvents and the preservative solutions.

### Example 4

### Cut Flower Applicable in the Present Invention

As cut flowers, a delphinium, a carnation, a gentian, a commelinaceae, a morning glory, a gerbera, a rose, a dahlia, a chrysanthemum, an amaryllis, an oxypetalum, an oncidium, an alstromeria, an eustoma russellianum, a dendrobium phalaenopsis and a globe amaranth were used. The petals of these various flowers were immersed in the hydrophilic organic solvent (ethanol) for not less than one day, after which they were immersed in the nonvolatile organic solvent (polypropylene glycol) as the preservative solution for not less than one day. Subsequently, the surface of the petals was washed with 1-butanol. Thus, sixteen types of processed cut flowers in total were produced.

As a result, the processed cut flowers of the delphinium, the carnation, the gentian, the commelinaceae, the morning glory, the gerbera, the rose, the dahlia, the alstromeria, the eustoma russellianum, the dendrobium phalaenopsis and the globe amaranth maintained qualities (shape, texture, color, etc.) similar to those of the natural state for not less than two weeks. As for other processed cut flowers of the chrysanthemum, the amaryllis, the oxypetalum and the oncidium, although slight color fading was observed, the shape of the petals were maintained even after two weeks, and therefore it was determined that they were excellent in maintaining the fresh state for a long period of time.

Similarly, the petals of the above sixteen types of cut flowers (delphinium, carnation, gentian, commelinaceae, morning glory, gerbera, rose, dahlia, chrysanthemum, amaryllis, oxypetalum, oncidium, alstromeria, eustoma russellianum, dendrobium phalaenopsis and globe amaranth) were processed using ethanol as the hydrophilic organic solvent, and a hardly-volatile organic solvent (2-methyl-2,4-pentanediol) as the preservative solution (without the washing step), so as to produce processed cut flowers (sixteen types). As a result, slight color fading was observed in some of them, but all the processed cut flowers maintained qualities similar to those of the natural state for not less than one week, and it was determined that they were excellent in maintaining the fresh state for a long period of time.

What is claimed is:

1. A method for preserving a cut flower by replacing tissue fluid of the cut flower by a preservative solution, the method including:
(1) a first replacement step in which the tissue fluid of the cut flower is replaced by a hydrophilic organic solvent; and
(2) a second replacement step in which the hydrophilic organic solvent that has replaced the tissue fluid in the cut flower is replaced by a preservative solution comprising an organic solvent that has an affinity for the hydrophilic organic solvent and is nonvolatile or hardly-volatile.

2. The method for preserving a cut flower according to claim 1,
wherein the preservative solution is at least one selected from the group consisting of 2-methyl-2,4-pentanediol, 2-ethyl-1,3-hexanediol, 2-[2-(2-ethylhexyloxy)ethoxy]ethanol, 3-methyl-1,5-pentanediol, 2-(2-ethylhexyloxy)ethanol and polypropylene glycol.

3. The method for preserving a cut flower according to claim 1,
wherein the hydrophilic organic solvent includes at least one liquid selected from the group consisting of ethanol, 1,4-dioxane, n-butyric acid, acetone and 1-propanol.

4. The method for preserving a cut flower according to claim 1, further including a washing step in which the surface of the cut flower having been subjected to the second replacement step is rinsed with a further hydrophilic organic solvent than said hydrophilic organic solvent.

5. A cut flower preservation kit that is usable to perform the method for preserving a cut flower according to claim 1, the kit including at least the hydrophilic organic solvent and the preservative solution.

6. A method for manufacturing a processed cut flower by replacing tissue fluid of a cut flower by a preservative solution, the method including:
(1) a first replacement step in which the tissue fluid of the cut flower is replaced by a hydrophilic organic solvent; and
(2) a second replacement step in which the hydrophilic organic solvent having replaced the tissue fluid in the cut flower is replaced by a preservative solution comprising an organic solvent that has an affinity for the hydrophilic organic solvent and is nonvolatile or hardly-volatile.

7. The method for manufacturing a processed cut flower according to claim 6,
wherein the preservative solution is at least one selected from the group consisting of 2-methyl-2,4-pentanediol, 2-ethyl-1,3-hexanediol, 2-[2-(2-ethylhexyloxy)ethoxy]ethanol, 3-methyl-1,5-pentanediol, 2-(2-ethylhexyloxy)ethanol and polypropylene glycol.

8. The method for manufacturing a processed cut flower according to claim 6,
wherein the hydrophilic organic solvent includes at least one liquid selected from the group consisting of ethanol, 1,4-dioxane, n-butyric acid, acetone and 1-propanol.

9. The method for manufacturing a processed cut flower according to claim 6, further including a washing step in which the surface of the cut flower having been subjected to the second replacement step is rinsed with a further hydrophilic organic solvent than said hydrophilic organic solvent.

10. A processed cut flower manufactured by the method for manufacturing a processed cut flower according to claim 6.
